(19) United States
(12) Patent Application Publication
(10) Pub. No.: US 2009/0287077 A1
(43) Pub. Date: Nov. 19, 2009

(54) SYSTEM FOR DETECTING FUNCTION OF INTESTINAL BARRIER AND METHOD FOR USING THE SAME

(75) Inventors: Linda Chia-Hui Yu, Taipei (TW); Jong-Kai Hsiao, Taipei (TW)

Correspondence Address: EDWARDS ANGELL PALMER & DODGE LLP, P.O. BOX 55874, BOSTON, MA, 02205, US

(73) Assignee: NATIONAL TAIWAN UNIVERSITY, Taipei (TW)

(21) Appl. No.: 12/388,694

(22) Filed: Feb. 19, 2009

(30) Foreign Application Priority Data
May 19, 2008 (TW) 097118335

(51) Int. Cl. A61B 5/055; A61K 49/00; C12Q 1/02; A61B 6/00

(52) U.S. Cl. 600/420; 424/9.1; 435/29; 600/431

(57) ABSTRACT

A system for detecting function of an intestinal barrier in a subject is provided, which includes: a contrast agent for being administered directly to an intestinal lumen of the subject, allowing a detecting device to detect in vivo or in vitro the signal indication produced by the contrast agent in the tested targets such as abdominal organs or a blood sample; and a determining device for determining intensity of the signal indication produced by the contrast agent in the tested targets after the administration of the contrast agent so as for the intensity of the signal indication after the administration of the contrast agent to indicate damage of the intestinal barrier in the subject. The present invention also provides a method of detecting function of the intestinal barrier in a subject by using the system of the present invention.

### BACKGROUND OF INVENTION

1. Field of Invention

The present invention relates to systems and methods for detecting function of an intestinal barrier in a subject, and more particularly, to a system and method for detecting function of a subject's intestinal barrier in vivo or in vitro.

2. Description of Related Art

The major functions of gastrointestinal tract are digestion and absorption of nutrients from food particles whereby the gastrointestinal tract is capable of serving as the first defense against orally ingested pathogenic microorganisms.

An intestinal barrier, consisting of a monolayer of epithelial cells connected by tight junctions, segregates the harmful substances in the intestinal lumen from systemic circulation and hence plays an important role in the physical defense of intestine. There are large numbers of commensals in the intestinal lumen, which constitute intestinal microflora. Once the intestinal barrier is damaged, the bacteria in the intestinal lumen enter systemic circulation, and reach other visceral organs. This phenomenon is called “bacterial translocation”. In addition, abnormal intestinal permeability, resulting in increased translocation of bacteria and antigenic products to visceral organs may lead to release of proinflammatory factors and infiltration of white blood cells and eventually cause damage of these visceral organs. Therefore, damage of an intestinal barrier is one of the important contributors to remote organ dysfunction, systemic inflammatory response, sepsis and multi-organ failure.

Pathological conditions due to loss of intestinal barrier function are often seen in bacterial infection, irritable bowel syndrome, food allergy, intestinal ischemia/reperfusion injury, intestinal obstruction, and cardiovascular diseases. Patients with severe dysfunction of the intestinal barrier often exhibit poor systemic circulation, such as septic shock and traumatic shock. When these patients have recovered from these symptoms and the ischemic visceral organs have been reperfused, these organs may be further injured by inflammatory factors and free radicals, which exacerbate tissue damage, in the newly returning blood. This phenomenon is called “ischemia-reperfusion injury”. The intestinal epithelial layer is sensitive to ischemia-reperfusion injury, which may result in damage to the morphology and function of the intestinal epithelial layer, for example, shortening of villi, destruction of villus apex, epithelial shedding, increased cell death, mitochondrial swelling, energy depletion, and production of reactive oxygen or nitrogen free radicals. Intestinal ischemia-reperfusion injury may also occur in other cases, for example, extracorporeal circulation, abdominal surgery, aortic aneurysm surgery, small intestine transplantation, strangulated hernia, necrotic entero-colitis in newborn infants etc.

The currently available techniques for detecting the function of the intestinal barrier are limited. The intestinal permeability has been determined by applying an enzyme probe, a fluorescene probe or a radio probe to the lumen surface or the serous membrane surface of an isolated intestinal segment in vitro; then detecting the probe penetrating across the barrier to the other side. The disadvantage of the technique is that it is necessary to isolate the intestinal tissue from its anatomical position and physiological environment, and the technique is mostly applied after the symptoms have occurred or aggravated.

“Sugar ingestion method” has been the only in vivo method used for detecting the function of the intestinal barrier in human subjects or animals so far. The method comprises the steps of orally administrating a mixed sugar solution to the tested subject and then measuring the differential amount of sugar excreted in the urine by high performance liquid chromatography, thereby evaluating the function of the intestinal barrier in the tested subject. Although the sugar ingestion method does not need to isolate the intestinal tissue from the surroundings, it is labor-consuming and time-consuming. In addition, since the sugar level in urine may be affected by renal tubular secretion, the interpretation of the result may be affected as well.

Magnetic resonance imaging (MRI) is usually used in detecting blood flow status in a tissue or an organ that has been changed in morphology for example due to tumor formation, luminal stenosis or distension. Detection of the anatomical position and status of pathological foci by MRI is usually performed by an intravenous injection of a contrast agent, a gadolinium (Gd)-containing contrast agent for example, to enhance T1- or T2-weighted image. The Gd-containing contrast agent is usually administered to the tested subject by a single intravenous injection at a dose of 2 to 20 ml (concentration: 0.5M).

A novel technique, called “MR enteroclysis”, has been used in intravenous illumination of intestinal wall so as to detect intestinal diseases such as tumor, stenosis and other motility-related diseases. An negative oral contrast agent based on super-paramagnetic particles has been used to enhance the image contrast ratio of GI tract lumen to other abdominal organs, or to distend the intestinal lumen to increase the surface area of intestinal mucosa so that the abscess or polyp can be visualized. However, there has been no method developed to evaluate the physiological function of intestine by MRI so far.

Therefore, it is still desired to develop a real-time, in vivo or in vitro system for evaluating the function of the intestinal barrier.

### SUMMARY OF INVENTION

The present invention relates to detection of the physiological function of intestine, rather than the anatomical structure of intestine, by using a contrast agent.

In one aspect of the present invention, a system is provided for detecting the function of the intestinal barrier in a subject, comprising:

a contrast agent for being administered directly to the intestinal lumen of the subject, rather than being administered intravenously to the subject as in the conventional method;

a detecting device for in in vivo detection of a signal indication produced by the contrast agent in visceral organs of the subject; and

a determining device for determining intensity of the signal indication produced by the contrast agent in the visceral organs of the subject after the administration of the contrast agent so as for the intensity of the signal indication after the administration of the contrast agent to indicate damage of the intestinal barrier in the subject.

According to the present invention, the contrast agent is a positive contrast agent, such as a gadolinium (Gd)-containing contrast agent, ferric chloride and an iodine-containing agent, wherein the Gd-containing contrast agent is preferred. The contrast agent is usually applied at a concentration of 0.05 to 1 M, preferably 0.1 to 0.3 M. The contrast agent is usually applied in an amount of 0.1 to 1 ml, preferably 0.3 to 0.6 ml.

The detecting device is used to detect the signal indication produced by the contrast agent in the multiple regions of the visceral organs (such as liver, kidney etc.) in the subject within 5 to 60 minutes, preferably 5 to 30 minutes (depending on which visceral organ is detected), after the administration of the contrast agent.

The determining device is used to determine the intensity of signal indication produced by the contrast agent in each of the multiple regions of the visceral organs of the subject after the administration of the contrast agent relative to that before the administration of the contrast agent, and to determine the mean value of the intensities of the signal indications so as for the mean value to indicate damage extent of the intestinal barrier of the subject. The system of the present invention is advantageous over the conventional detection system, in which the detection is performed only at a single position for a single time point, since the variation in the former caused by the background values of the tested subject can be eliminated.

According to the present invention, the detecting device can be a MR imager.

In another aspect of the present invention, a system is provided for detecting the function of the intestinal barrier in a subject, comprising:

a contrast agent for being administered directly to the intestinal lumen of the subject, rather than being administered intravenously to the subject as in the conventional method;

a detecting device for in vitro detection of a signal indication produced by the contrast agent in a blood sample taken from the subject; and

a determining device for determining intensity of signal indication produced by the contrast agent in the blood sample after the administration of the contrast agent so as for the intensity of the signal indication after the administration of the contrast agent to indicate damage of the intestinal barrier in the subject.

According to the present invention, the blood sample can be whole blood, plasma or serum.

According to the present invention, the contrast agent is a positive contrast agent, such as a gadolinium-containing contrast agent, ferric chloride and an iodine-containing agent, wherein the gadolinium-containing contrast agent is preferred. The contrast agent is usually applied at a concentration of 0.05 to 1 M, preferably 0.1 to 0.3 M. The contrast agent is usually applied in an amount of 0.1 to 1 ml, preferably 0.3 to 0.6 ml.

The detecting device is used to detect the signal indication produced by the contrast agent in the blood sample taken from the tested subject before the administration of the contrast agent and within 5 to 60 minutes, preferably 5 to 30 minutes, after the administration of the contrast agent.

According to the present invention, the detecting device can be a MR imager.

According to the present invention, the determining device is used to determine a concentration of the contrast agent in the blood sample of the subject according to the intensity of the signal indication produced by the contrast agent in the blood sample, so as for the concentration to indicate damage extent of the intestinal barrier of the subject.

In further aspect of the present invention, a method is provided for detecting the function of the intestinal barrier in a subject by using the systems of the present invention.

### BRIEF DESCRIPTION OF THE INVENTION

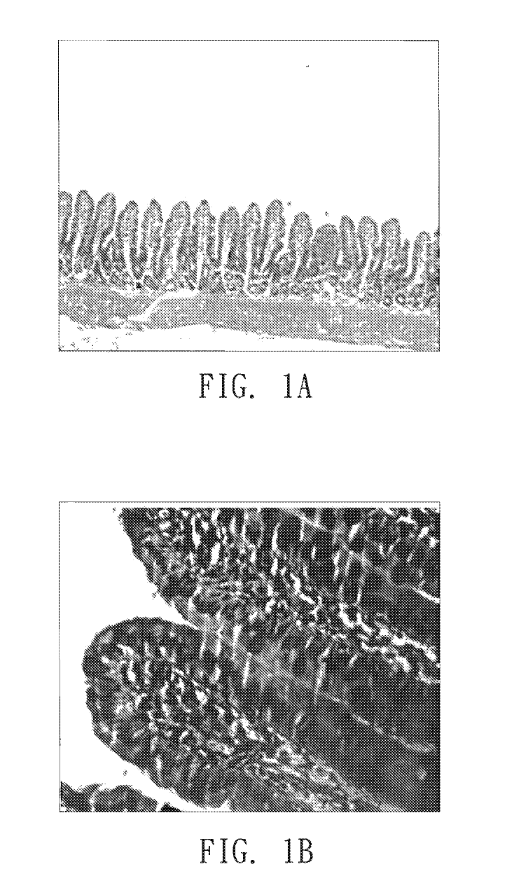

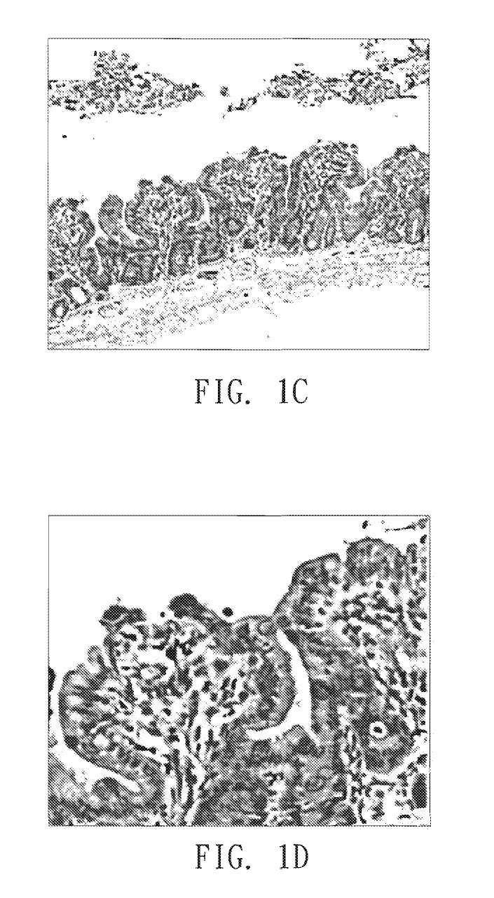

FIGS. 1A to 1D are photographs showing the jejunal tissues of a rat of the sham-operation control group (hereafter referred to as “CON”) and a rat of ischemia-reperfusion group (hereafter referred to as “I/R”). FIG. 1A is a photograph showing the normal jejunal tissue of a CON rat and FIG. 1B is a magnified image of FIG. 1 (amplifying power: FIG. 1A, 100×; FIG. 1B, 400). FIG. 1C is a photograph showing the damaged jejunum, including destruction of villus apex, epithelial shedding, shortening and widening of villi etc., of an I/R rat. FIG. 1D is a magnified image of FIG. 1C (amplifying power: FIG. 1C, 100×; FIG. 1D, 400) (n=6 to 8 per group).

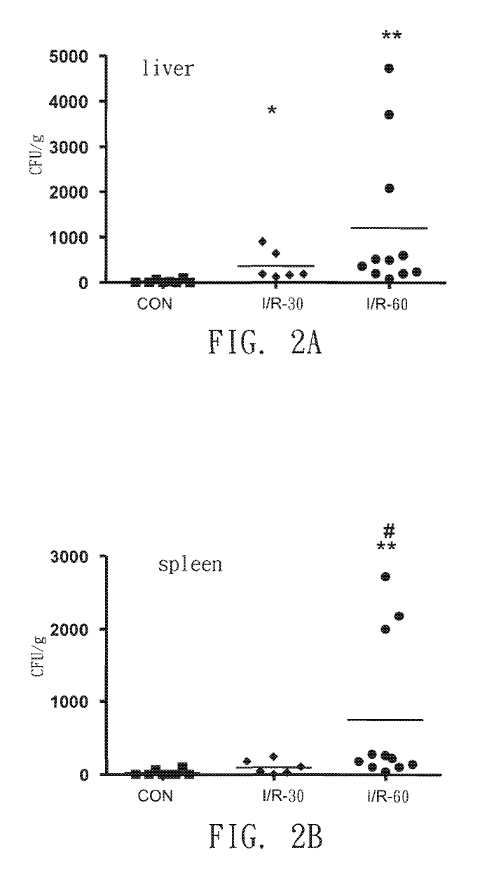

FIGS. 2A and 2B are graphs respectively showing the colony forming unit (expressed in CFU/g) of the intestinal bacteria translocated to the liver (FIG. 2A) and the spleen (FIG. 2B) in CON rats, I/R-30 rats and I/R-60 rats, wherein I/R-30 rats refer to a group of rats that were subjected to a 20-minute SMA closure followed by a 30-minute reperfusion and I/R-60 rats refers to a group of rats subjected to a 20-minute SMA closure followed by a 60-minute reperfusion. The values reported in the figures are the mean values for all groups. (*: p<0.05 relative to CON; **: p<0.01 relative to CON; #: p<0.05 relative to I/R-30) (n=6 to 10/group).

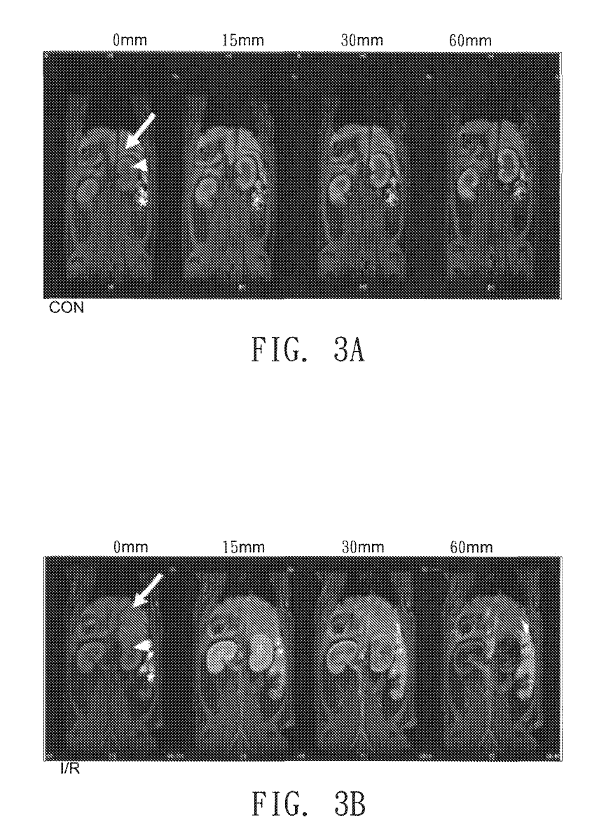

FIGS. 3A and 3B are photographs respectively showing the MRI images of the abdominal organs in a CON rat and an I/R rat, before reperfusion and at different time points after reperfusion (namely, after the administration of the Gd-containing contrast agent to the jejunal pouch; since the contrast agent was administered as soon as reperfusion). In these two figures, the symbols “arrow”, “arrowhead” and “star” indicate the positions of the liver, spleen and fastened jejunum respectively; “0 min” on x-axis refers to the time point when the MRI measurement was made before the administration of the Gd-containing contrast agent to the jejunal pouch; and “15 min., 30 min. and 60 min.” on x-axis respectively refer to the time points when the MRI measurements were made at 15, 30 and 60 minutes after the administration of the Gd-containing contrast agent to the jejunal pouch. (n=4 to 6/group).

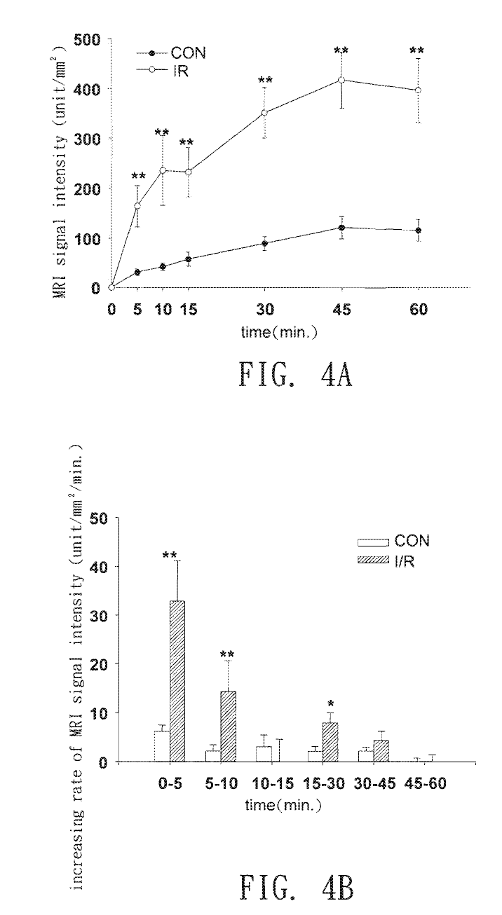

FIG. 4A is a graph showing the MRI signal intensity (unit/min.) of the liver before reperfusion and at different time points (min) after reperfusion in CON rats and I/R rats. FIG. 4B is a graph showing the increasing rate of the MRI signal intensity of the liver at different stages after reperfusion in CON rats and I/R rats. Each value reported in FIGS. 4A and 4B indicates the mean±SEM (*: p<0.05 relative to CON; **: p<0.01 relative to CON) (n=4 to 6/group).

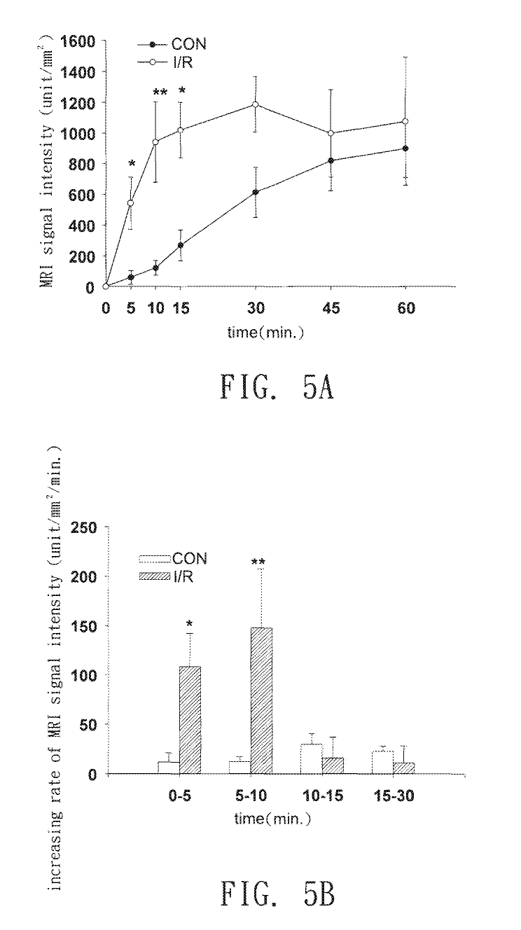

FIG. 5A is a graph showing the MRI signal intensity (unit/min.) of the kidney before reperfusion and at different time points (min.) after reperfusion in CON rats and I/R rats. FIG. 5B is a graph showing an increase in the rate of the MRI signal intensity of the kidney at different stages after reperfusion in CON rats and I/R rats. Each value reported in FIGS. 5A and 5B indicates the mean±SEM (*: p<0.05 relative to CON **: p<0.01 relative to CON) (n=4 to 6/group).

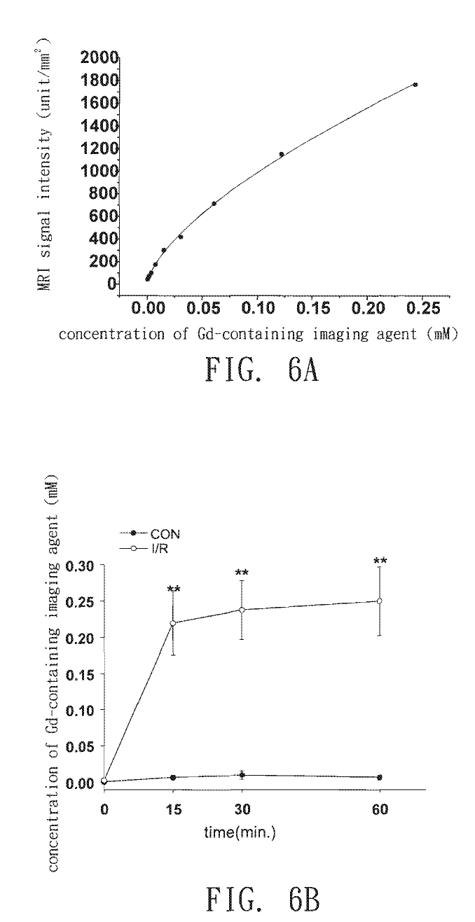

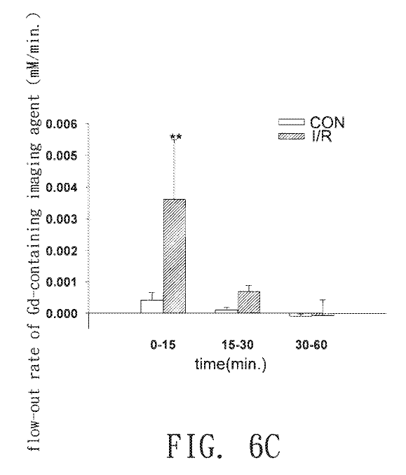

FIG. 6A is a graph showing the standard curve of MRI signal intensity (unit/mm²) versus the concentration (mM) of the Gd-containing contrast agent. FIG. 6B is a graph showing the concentration (mM) of the Gd-containing contrast agent in the plasma samples taken before reperfusion and at different time points (min) after reperfusion in CON rats and I/R rats. FIG. 6C shows the flow-out rate of the Gd-containing contrast agent from the intestinal lumen to systemic circulation at different stages after reperfusion in CON rats and I/R rats. Each value reported in FIGS. 6A to 6C indicates the mean±SEM (*: p<0.01 relative to CON) (n=6/group).

### DETAILED DESCRIPTION OF THE INVENTION

Next, the present invention is further illustrated by the following specific embodiments with reference to the above figures. The advantages and effects of the present invention can be easily understood by the persons skilled in the art from the disclosure of this specification.

### Definition of Terms:

The term “contrast agent” herein, also referred to as “imaging agent”, is used in enhancement of tissue imaging or change of image contrast ratio in MRI scan or CT scan when administered to the human body. Stomach, intestine, biliary duct, urinary system, blood vessels, which cannot be visualized by X-ray, can be visualized by the aid of a contrast agent. The present invention not only augments the image contrast ratio of the tissue image as the prior art but also allows the assessment of the damage extent of the intestinal barrier by administrating a contrast agent directly to the intestinal lumen of the tested subject and then detecting the signal indication produced by the contrast agent in the visceral organs or the blood samples of the tested subject.

There are two types of contrast agents, one is the positive contrast agent and the other is the negative contrast agent. The positive contrast agent enhances the brightness of an image; the examples thereof include a gadolinium (Gd)-containing contrast agent, ferric chloride, an iodine-containing agent. The negative contrast agent reduces the brightness of an image; the examples thereof include air, carbon dioxide, water, a super-paramagnetic contrast agent. MRI scan usually utilizes a Gd-containing contrast agent or other positive contrast agents, while CT scan usually utilizes an iodine-containing agent, which is a positive contrast agent, to enhance the brightness of the image. The present invention preferably utilizes a Gd-containing contrast agent, which is non tissue-specific, non-ionic, low toxic, low allergic, and mainly used in MRI scan or MRA (magnetic resonance angiography). Gd compounds can be entirely excreted form kidney within a period of 24 hours. The contrast agent is administrated to the intestinal lumen by intravenous injection in the previous methods, while directly administrated to the intestinal lumen by using an endoscope for example in the present invention.

The term “detecting device” herein refers to the device that can detect the signal indication produced by the contrast agent in a visceral organ or a blood sample. The detecting devices include, but are not limited to, MR Imagers and CT scanners.

The term “ischemia” herein refers to a condition of a restriction in blood supply, which results in insufficient supply of oxygen and glucose. The term “reperfusion” herein refers to a condition that a restoration of circulation occurs in the local tissue after a period of ischemia, which may cause free radical-mediated injury (also called “ischemia-reperfusion injury”).

### EXPERIMENT EXAMPLE

### Experimental Procedures

Male Wistar rats weighed 250 g to 300 g were used in this experiment. The rats were randomly divided into 2 groups: sham-operation control group (CON) and ischemia/reperfusion group (I/R). Before the experiment, the rats were fasted overnight but allowed to drink water ad lib. The experiment was conducted in a sterile environment. The rats in the two groups were anethesized by intraperitoneal injection of ethyl urethane (1.2 g/kg), and then subjected to abdominal median incision. A jejunal pouch of 10 cm was made at 10 cm from the end of suspensory ligament of duodenum. A 1 ml syringe with a PE-10 catheter was intubated to one end of the jejunal pouch and 0.5 ml of Krebs buffer solution was slowly injected into the pouch from the syringe. The formula of Krebs buffer solution was as follows: 115 mmol/L of NaCl, 8 mmol/L of KCl, 1.25 mmol/L of Ca Cl₂, 1.2 mmol/L of MgCl₂, 2.0 mmol/L of KH₂PO₄ and 25 mmol/L of NaHCO₃, with a pH of 7.33 to 7.37. For the I/R rats, superior mesenteric artery (SMA) was isolated and its root, which is branched from abdominal aorta, was clamped with a sterile atraumatic artery clamp (0.6 cm) to close SMA for 20 minutes, and then the clamp was released to allow reperfusion for 1 hour. The CON rats were subjected to abdominal median incision but not subjected to SMA closure. Thereafter, the tissue samples were taken from the rats of these 2 groups for histologic examination and analysis of bacterial translocation (as shown below).

### Histologic Pathology

In the last step of the experiment, a jejunal segment was taken from the jejunal pouch, fixed with 4% paraformaldehyde (pH 7.4, in PBS), and embedded in paraffin with glandular tube to villi axis in an appropriate orientation. The slice of 4 μm thick was taken and deparrafinized in xylene and graded ethanol, and then stained with hematoxylin and eosin (H &E) (Sigma-Aldrich Co., MO, USA). The slice was observed by a low power microscope.

### Analysis of Bacterial Translocation

After surgical operations, the spleen and liver were cut from each rat of the two groups and weighed. The spleen and the liver were respectively homogenized in a plastic bottle and the two homogenates were adjusted to a concentration of 0.1 g/ml with PBS. Each homogenate was added to both a fresh blood agar plate and a MacConkey agar plate (100 μl/plate) and the plates were incubated at 37° C. overnight to examine the existence of all bacteria and G(−) bacteria in the homogenates. The colony forming unit (CFU) was calculated for each homogenate and expressed in CFU per g of tissue (CFU/g).

### Magnetic Resonance Imaging (MRI)

In order to assess intestinal permeability, a contrast agent was directly administered to the lumen of the jejunal pouch. Then, abdominal MRI was performed by using a clinical 1.5T MR system (Signa excite, GE Healthcare, USA), to measure the signal indication produced by the contrast agent in the liver and the kidney of the tested rats. The flow-out rate of the Gd-containing contrast agent flowing from the intestinal lumen to the liver via hepatic portal veins and to the kidney via systemic circulation (equal to the flow-out rate of the Gd-containing contrast agent from the lumen surface to the serous membrane surface of intestine) is used as an indicator of intestinal permeability.

A Gd-containing contrast agent (Omniscan™, MW 574, GE Healthcare Ireland, Cork, Ireland) was slowly injected into the jejunal pouch at an amount of 0.5 ml (0.5 M) immediately after the release of the artery clamp in I/R rats. In CON rats, the Gd-containing contrast agent was injected into the jejunal pouch after sham operation. Thereafter, the rats of the 2 groups were scanned by MRI. The rats were placed in a self-made RF coil (inner diameter: 6 cm). Two-dimensional, T1-weighted, fast spin-echo pulse sequences provided by FSE-XL/90 were used (TR/TE=140/4.2 ms, echo train length=1/1, bandwidth=65.4 kHz), with an image width of 1.4 mm and an interval of 0.03 mm. The field of vision (FOV) was 12×8.4 cm² so that the abdominal organ of the rat can be visualized. The total scan time lasted for 1 minute and 4 seconds.

### Data Analysis

### Image Data

The rats were scanned by MRI at 0, 5, 10, 15, 30, 45 and 60 min after reperfusion (namely, after the administration of the Gd-containing contrast agent). Thereafter, the signal indication produced by the contrast agent in the circled regions of the liver, the left kidney and the right kidney (3 circled regions with a surface area in each of 38.83 mm²) at each time point was calculated by a determining device. The circled regions covered the left lobe, right upper lobe and right lower lobe of the liver, and the upper, middle and lower parts of the kidney. The average intensity of signal indication at each time point for each organ was calculated and expressed in unit/mm², and was further corrected of background variation by subtracting the average intensity of signal indication at 0 min therefrom. The flow-out rate of the Gd-containing contrast agent flowing from the lumen to the serous membrane of intestine was shown by the increase in the intensity of signal indication in the liver and the kidney per minute (unit/mm²/min).

### Blood Data

In order to determine the rate of the Gd-containing contrast agent transferred from jejunal lumen into systemic circulation, a PE-50 catheter was intubated into left femoral artery before the surgical operation. Blood samples (0.5 ml per sample) were taken from the rats at 0, 15, 30 and 60 min after reperfusion and the Gd-containing contrast agent was injected into the jejunal pouch of the rats. Each of the whole blood sample was centrifuged at 3500×g, 37° C. for 15 min to obtain a plasma sample. In another aspect, a solution of the Gd-containing contrast agent at the known concentration (0.5 M) was serially diluted with a mixture of the plasma from 3 CON rats to obtain a series of standard solutions of the Gd-containing contrast agent. All plasma samples (80 μl) and all standard solutions (80 μl) were respectively collected in test tubes. The test tubes were placed on a 96-place rack in a water bath, and then were subjected to MRI scan. The intensity of signal indication of each plasma sample and of each standard solution was calculated by using a determining device to circle a region of 4 mm² in MRI image and to calculate the intensity of signal indication in the region (mm²). Each calculation was repeated twice and the intensity of signal indication was expressed in unit/mm². The intensity of signal indication of the standard solutions of the Gd-containing contrast agent was plotted against the concentration thereof to establish a standard curve. The concentration of the Gd-containing contrast agent in the plasma samples at each time point was calculated, using the standard curve. The flow-out rate of the Gd-containing contrast agent flowing from intestinal lumen to systemic circulation at different stages of reperfusion was calculated and expressed in mM/min.

### Statistic Analysis

All results were expressed in mean±SEM, and all MRI data were compared by analysis of variance (ANOVA), followed by Student-Newman-Keul test. For bacterial translocation analysis, non-parameter Mann-Whitney U-Test was used to compare the medians of the experimental data. The statistic significance of p value is <0.05.

### Results

### Ischemia and Reperfusion (I/R) Induced Morphologic Damage and Functional Defect

The rats subjected to 20 minute SMA closure and 60 minute reperfusion showed damage in the jejunal mucosal tissue, including destruction of villus apex and epithelial shedding (FIGS. 1C and 1D) while the intestinal villi observed in the sham-operation control group remained intact (FIGS. 1A and 1B). In addition, shortening and widening of villi were observed in the jejunum of I/R rats (FIGS. 1C and 1D).

In order to assess the damage extent of the intestinal barrier, the amount of the live bacteria in the liver and spleen which indicates the extent of intestinal bacterial translocation was evaluated. As shown in FIG. 2A, the I/R rats subjected to ischemia followed by 30 min reperfusion (I/R-30) or 60 min of reperfusion (I/R-60) showed significant increase in CFU per gram of hepatic tissue (363.3±133.2 and 1204±484.8 CFU/g, respectively) when compared with the CON rats (22.5±13.33 CFU/g). The I/R rats also showed increased CFU values in the spleen when compared with the CON rats, as shown in FIG. 2B. The results suggest that the extent of the intestinal bacterial translocation to the liver and kidney depends on the period of reperfusion.

Increased MRI Signal Intensity in the Abdominal Organs Shown in I/R Rats Compared with CON Rats

The present invention develops a novel use of MRI in real-time detection of the change in intestinal permeability in vivo. The contrast agent containing an inert Gd compound (MW=574) was injected into the jejunal pouch of an I/R rat and the jejunum of a CON rat as soon as reperfusion. The rats of these two groups were subjected to abdominal MRI scan before reperfusion (i.e. “0 min.” in FIGS. 3A and 3B) and at 15, and 60 min. after reperfusion (i.e. “15 min.”, “30 min.” and “60 min.” in FIGS. 3A and 3B), so as to evaluate the extent of the contrast agent transferred from the intestinal lumen to the abdominal organs, including that entering the liver via hepatic portal vein and that entering the kidney via systemic circulation.

The MRI images of the liver and the kidney of the CON rat, as shown in FIG. 3A, provided a baseline of the extent of the Gd-containing contrast agent transferred from the intestinal lumen to the abdominal organs. As shown in FIG. 3B, in the I/R rat, the signal intensity and the brightness of the liver MRI image taken at 15 min. was higher than that taken at 0 min. and such high signal intensity lasted for 60 minutes after reperfusion. The signal intensity of the kidney MRI image taken at 15 min. was higher than that taken at 0 min, but the signal intensity began to decrease at 30 min after reperfusion. The decrease in signal intensity of the kidney image taken at 30 min may be owing to that accumulation of the contrast agent in the kidney led to higher T2 effect on MRI, which resulted in decreased signal intensity of T1-weighted image.

### Quantifying the MRI Signal Intensity of the Liver and the Kidney in Con Rats and I/R Rats

The MRI signal intensity of the liver and the kidney of CON rats and I/R rats was measured by an image analyzer and corrected for background variation by subtracting the background signal intensity of the liver or the kidney therefrom.

As shown in FIG. 4A, the MRI signal intensity of the liver in I/R rats was significantly higher than that in CON rats; in addition, the MRI signal intensity increased by 5 times after 5 minutes of reperfusion and maintained high level until the end of reperfusion. Kinetic analysis, as shown in FIG. 4B, revealed that the increasing rate of the MRI signal intensity of the liver in I/R rats occurred reached maximum in the early stage of 0 to 5 minutes of reperfusion, and then decreased.

Similar to the liver, the MRI signal intensity of the kidney in I/R rats was significantly higher than that in CON rats (FIG. 5). Kinetic analysis, as shown in FIG. 5B, revealed that the increasing rate of the MRI signal intensity of the kidney in I/R rats was 10 times more than that in CON rats during the stages of 0 to 5 min and the stage of 5 to 10 min of reperfusion. In addition, the increasing rate in I/R rats reached maximum at 30 min, then decreased, which suggests that high concentration of the contrast agent accumulated in the kidney led to increase in T2-shortening effect.

Significantly Higher Plasma Level of Gd in I/R Rats when Compared with Con Rats.

In order to quantify the amount of the Gd-containing contrast agent seeped out of the intestinal barrier to systemic circulation, the blood samples were taken before reperfusion and at different time points after reperfusion. The MRI signal intensity in plasma isolated from each of the blood samples was measured. In addition, a solution of the Gd-containing contrast agent at the known concentration was serially diluted with normal plasma to obtain a series of standard solutions. The MRI signal intensity in these standard solutions was measured and plotted against the concentration thereof to establish a standard curve (FIG. 6A). The concentration of the Gd-containing contrast agent in the plasma was calculated, using the standard curve. After 15 minutes of reperfusion, the concentration of the Gd-containing contrast agent in the plasma of I/R rats was 0.220±0.044 mM, which was significantly higher than that in the plasma of CON rats (0.006±0.004 mM) (FIG. 6B). In addition, the flow-out rate (mM/min) of the Gd-containing contrast agent in I/R rats was significantly higher than that in CON rats during the stage of 0-15 minutes of reperfusion.

### CONCLUSION

The above experimental results have proved that the function of intestinal barrier relates to the MRI signal intensity of a Gd-containing contrast agent in the liver or kidney after a contrast agent is directly administered into the intestinal lumen. In addition, the bacterial translocation due to mesenteric ischemia and reperfusion injury relates to the intestinal permeability, which depends on the function of intestinal barrier. Therefore, in case that alteration in intestinal barrier function is suspected or observed, the intestinal barrier function or intestinal permeability can be detected or monitored by the in vivo or in vitro method of the present invention, so that the alteration of intestinal barrier function can be controlled in time and the diseases caused by bacterial translocation from intestinal lumen to other visceral organs can be prevented.

The above experiment is merely an illustration of the system according to the present invention but should not be considered as a limitation thereto. The person skilled in the art can make an alteration or modification on the present invention without departing from the spirit and scope of the present invention. The patent protection scope of the present invention is defined by the following claims.

1. A system for detecting function of an intestinal barrier in a subject, comprising:
a contrast agent for being administered directly to an intestinal lumen of the subject;
a detecting device for in vivo detection of a signal indication produced by the contrast agent in visceral organs of the subject; and
a determining device for determining intensity of the signal indication produced by the contrast agent in the visceral organs of the subject after the administration of the contrast agent, so as for the intensity of the signal indication after the administration of the contrast agent to indicate damage of intestinal barrier in the subject.

2. The system according to claim 1, wherein the detecting device is used to detect the signal indication produced by the contrast agent in multiple regions of the visceral organs of the subject within 5 to 60 minutes after the administration of the contrast agent.

3. The system according to claim 2, wherein the determining device is used to determine the intensity of the signal indication in each of the multiple regions of the visceral organs of the subject after the administration of the contrast agent relative to that before the administration of the contrast agent.

4. The system according to claim 3, wherein the determining device is further used to determine the mean value of the intensities of the signal indications produced by the contrast agent in the multiple regions of the visceral organs of the subject, so as for the mean value to indicate damage extent of the intestinal barrier of the subject.

5. The system according to claim 1, wherein the contrast agent is used at a concentration of 0.05 M to 1 M.

6. The system according to claim 1, wherein the contrast agent is used in an amount of 0.1 to 1 ml.

7. The system according to claim 1, wherein the contrast agent is a positive contrast agent.

8. The system according to claim 1, wherein the contrast agent is a gadolinium-containing contrast agent.

9. The system according to claim 1, wherein the detecting device is a magnetic resonance imager.

10. A system for detecting function of an intestinal barrier in a subject, comprising:
a contrast agent for being administered directly to intestinal lumen of the subject;
a detecting device for in-vitro detection of a signal indication produced by the contrast agent in a blood sample taken from the subject; and
a determining device for determining intensity of the signal indication produced by the contrast agent in the blood sample after the administration of the contrast agent, so as for the intensity of the signal indication produced by the contrast agent after the administration of the contrast agent to indicate damage of the intestinal barrier in the subject.

11. The system according to claim 10, wherein the detecting device is used to detect the intensity of the signal indication produced by the contrast agent in the blood sample within 5 to 60 minutes after the administration of the contrast agent.

12. The system according to claim 10, wherein the determining device is used to determine a concentration of the contrast agent in the blood sample of the subject according to the intensity of the signal indication produced by the contrast agent in the blood sample, so as for the concentration to indicate damage extent of the intestinal barrier of the subject.

13. The system according to claim 10, wherein the contrast agent is used at a concentration of 0.05 M to 1 M.

14. The system according to claim 10, wherein the contrast agent is used in an amount of 0.1 to 1 ml.

15. The system according to claim 10, wherein the contrast agent is a positive contrast agent.

16. The system according to claim 10, wherein the contrast agent is a gadolinium-containing contrast agent.

17. The system according to claim 10, wherein the detecting device is a magnetic resonance imager.

18. A method for detecting function of an intestinal barrier in a subject by using the system of claim 1, comprising the steps of:
administering a contrast agent directly to an intestinal lumen of the subject;
using a detecting device to detect in vivo a signal indication produced by the contrast agent in visceral organs of the subject; and
using a determining device to determine intensity of the signal indication produced by the contrast agent in the visceral organs of the subject after the administration of the contrast agent, so as for the intensity after the administration of the contrast agent to indicate damage of the intestinal barrier in the subject.

19. A method for detecting function of an intestinal barrier in a subject by using the system of claim 10, comprising the steps of:
administering a contrast agent directly to an intestinal lumen of the subject;
using a detecting device to detect in-vitro a signal indication produced by the contrast agent in a blood sample taken from the subject; and
using a determining device to determine intensity of the signal indication produced by the contrast agent in the blood sample after the administration of the contrast agent, so as for the intensity of the signal indication produced by the contrast agent to indicate damage of the intestinal barrier in the subject.
